(19) United States
(12) Patent Application Publication
(10) Pub. No.: US 2004/0127867 A1
(43) Pub. Date: Jul. 1, 2004

(54) Absorbent article system

(76) Inventors: Thomas Walter Odorzynski, Green Bay, WI (US); Richard Louis Underhill, Neenah, WI (US); Cornelius Jacobus Bosselaar, Appleton, WI (US); Stephen Bradford Cook, Appleton, WI (US); David Arthur Fell, Neenah, WI (US)

Correspondence Address: KIMBERLY-CLARK WORLDWIDE, INC., 401 NORTH LAKE STREET, NEENAH, WI, 54956

(21) Appl. No.: 10/335,788

(22) Filed: Dec. 31, 2002

(51) Int. Cl. A61F 13/15; A61F 13/20

(52) U.S. Cl. 604/359

(57) ABSTRACT

A method for providing a system of absorbent articles including offering a chassis primarily performing a base function, wherein the chassis is an absorbent article; and offering first and second enhancement products, wherein the chassis and the first and second enhancement products are adapted such that the first and second enhancement products may be coupled to the chassis. Further, a method for educating a consumer with regard to various conditions requiring an absorbent article including providing first and second packages, each including a description of a condition and an enhancement product adapted to address the conditions; allowing the consumer to learn about the conditions and what may be used to address the conditions; and providing a third package including a chassis and description of a base condition, such that the chassis may be combined with the first and/or second enhancement products.

### BACKGROUND

A large number of improvements to absorbent articles such as diapers, feminine care products, and incontinence products have been identified and developed, but not commercially exploited. A great deal of time and money has been expended developing features from consumer concept studies to consumer preference tests. Although many of these product improvements are readily received by consumers, they have not been commercialized.

Limited add-on products for some absorbent articles such as disposable diapers have been sold commercially with little or no success, e.g. liner doublers.

### SUMMARY OF THE INVENTION

The problem with many of these improvements is benefit cost. Incorporating some is of these improvements into every absorbent article designed for daily use would be cost-prohibitive. For example, consumers may desire more absorbency in an absorbent product. More absorbency, however, equates to a higher cost per product. A one cent cost increase per product is generally considered significant. One cent, however, does not purchase a large amount of super-absorbent or absorbent fluff. Likewise, add-on improvements that do not modify current materials of construction may be difficult to deliver in every absorbent article for less than one cent per absorbent article.

Other improvements may not be commercially exploited because they significantly alter the product form. Some improvements may make the product too thick; others may radically change the product's packaging requirements. Finally, other improvements may not be commercially exploited because they may not be appropriate for all absorbent articles. For example, an improvement directed toward a skin care benefit may be more applicable to consumers with sensitive skin or a skin condition such as diaper rash. In another example, some consumers may prefer to control the amount of lotion applied to an absorbent article, or whether lotion is applied at all, rather than having the lotion pre-applied to the absorbent article.

To solve these problems, a new absorbent article system is required. The elements of the absorbent article system are generally a primary absorbent article or chassis, very similar or identical to products currently available, and secondary or enhancement products that are sold separately. Such a system allows a consumer to customize the primary absorbent article to meet the consumer's individual needs. Each enhancement product may include a description of the condition the enhancement product is used to address. As a result, the enhancement products may be used to educate the consumer.

The present invention overcomes the problems by providing a method for providing a system of absorbent articles, the method including offering a chassis primarily performing a base function, wherein the chassis is an absorbent article; offering a first enhancement product primarily performing a first enhanced function; and offering a second enhancement product primarily performing a second enhanced function, wherein the chassis and the first and second enhancement products are adapted such that the first and second enhancement products may be coupled to the chassis, and wherein the second enhanced function is different from the first enhanced function.

The present invention also provides a method for educating a consumer with regard to various conditions requiring an absorbent article, the method including providing a first package including a description of a first condition allowing the consumer to learn about the first condition and what may be used to address the first condition, wherein the first package includes a first enhancement product adapted to address the first condition; providing a second package including a description of a second condition allowing the consumer to learn about the second condition and what may be used to address the second condition, wherein the second package includes a second enhancement product adapted to address the second condition, and wherein the second condition is different from the first condition; and providing a third package including a description of a base condition allowing the consumer to learn about the base condition and what may be used to address the base condition, wherein the third package includes a chassis adapted to address the base condition and adapted such that the chassis may be combined with the first and/or second enhancement products, and wherein the base condition is different from the second condition.

The present invention also provides a system for addressing various conditions requiring an absorbent article, the system including an absorbent chassis adapted to absorb a bodily fluid; a first enhancement product adapted to perform a first enhanced function, wherein the chassis and the first enhancement product are adapted such that the first enhancement product may be coupled to the chassis; and a second enhancement product adapted to perform a second enhanced function, wherein the chassis and the second enhancement product are adapted such that the second enhancement product may be coupled to the chassis, and wherein the second enhanced function is different from the first enhanced function.

The present invention also provides a method for a consumer to customize an absorbent article to address a condition, the method including selecting a base absorbent article; examining a plurality of packages, each containing an enhancement product; selecting the enhancement product adapted to address the condition; and combining the enhancement product and the base absorbent article.

The present invention also provides a method for selling a customizable absorbent article system, the method including displaying on a store shelf a chassis, wherein the chassis is an absorbent article; and displaying, adjacent to the chassis, a plurality of different enhancement products, wherein the chassis and each enhancement product are adapted such that each enhancement product may be coupled to the chassis.

The present invention provides several attributes that are advantageous. An absorbent article system in which consumers may customize a basic absorbent article through the use of supplementary products enhances the efficiency of the absorbent-article-using process, helps to better care for the user, and may be more cost effective. Benefits of such a system to a business include a reduction of absorbent article machine capital expenditure, optimized manufacturing, more rapid evaluation and introduction of product improvements in the form of supplementary products, and potentially higher margins for supplementary products.

Other objects and advantages of the present invention will become more apparent to those skilled in the art in view of the following description and the accompanying drawings.

### BRIEF DESCRIPTION OF THE DRAWINGS

FIG. 1 is a schematic representation of an absorbent article system of the present invention.

FIG. 2 is a schematic representation of an exemplary toilet training system of the present invention.

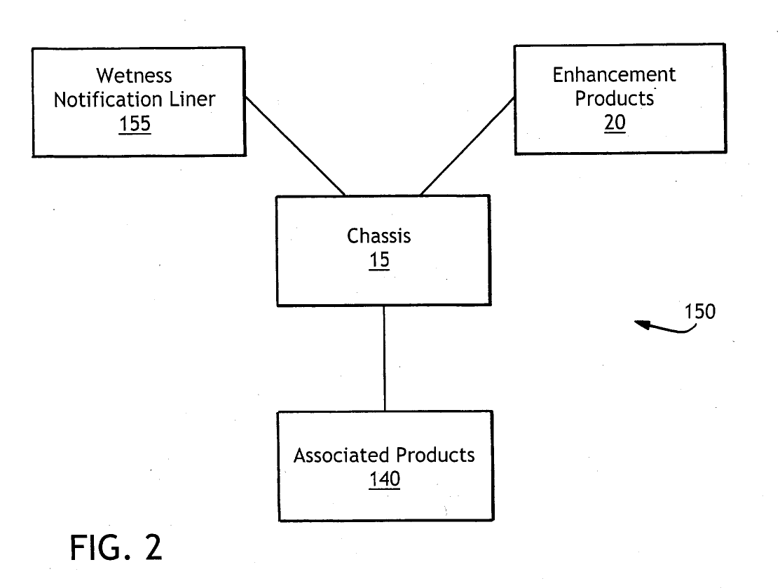

### DETAILED DESCRIPTION OF THE PREFERRED EMBODIMENTS

The invention described herein is a system of a primary absorbent article with secondary enhancement products. Such a system allows a consumer to customize the primary absorbent article to meet the consumer's individual needs.

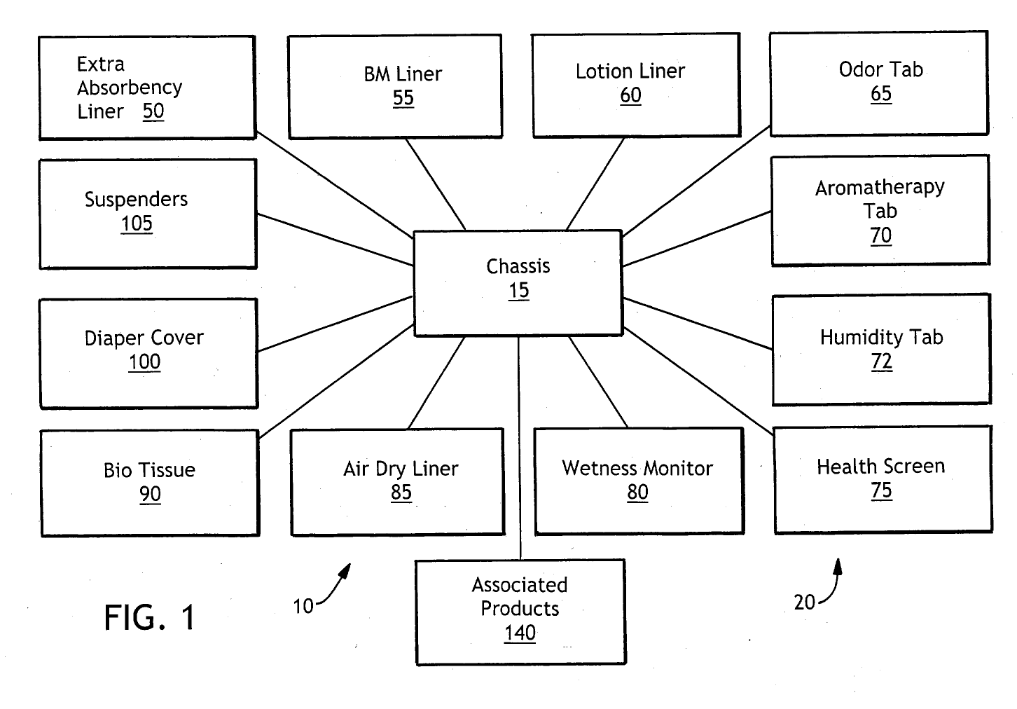

FIG. 1 illustrates an example of an absorbent article system 10 of the present invention. The system 10 includes a primary absorbent article or chassis 15 and a number of secondary or enhancement products 20. The absorbent article system 10 may include any type of absorbent article at its core, including, but not limited to, a diaper, a training pant, a swim pant, a youth pant, a feminine hygiene product, an incontinence product, or the like. For purposes of illustration, and not for purposes of limitation, the absorbent article system 10 is described as it would apply to a diaper. The same system, however, may be described using another suitable absorbent article.

The absorbent article system 10 includes a chassis 15 that may be very similar or identical to absorbent article products available to consumers. The chassis 15 is used to treat a base condition, typically the need for absorbency. The base function performed by the chassis 15 is typically waste collection and absorption. In the case of a diaper, the chassis 15 is a diaper that may be used on its own as a standard diaper. Again in the case of a diaper, the chassis 15 or basic diaper component of the absorbent article system 10 is preferably a more premium-level disposable diaper, but may also be a lower-end, non-premium diaper, or any other suitable disposable diaper. The term “basic” in this instance implies that the diaper performs the typical and expected duties of a disposable diaper including a certain level of urine and fecal absorbency and containment, along with an acceptable level of comfort, fit, and skin health. For example, a diaper fitting the basic classification may include a breathable cloth-like outer cover, mechanical fasteners, superabsorbent material and/or fluff absorbents, stretch ears, surge, feces or bowel movement (BM) containment flaps, gentle leg elastics, and a back waist elastic system. This type of product is generally sold as a premium diaper.

The chassis or basic diaper 15 will meet the needs of most consumers, and performs better than, or at least as well as, other premium disposable diapers.

The absorbent article system 10 also includes a variety of secondary or enhancement products 20. Each of these enhancement products 20 performs at least one primary function such as humidity or odor control. Enhancement products 20 are designed to complement the chassis 15 by being disposed within or around the chassis 15, or otherwise coupled to the chassis 15, as described in more detail herein. The enhancement products 20 deliver benefits that allow the consumer to customize the chassis 15 to the consumer's needs.

While the enhancement products 20 may take any suitable form or function, a variety of examples are described herein for use in a diaper-based system. Similar enhancement products 20 may be used in systems based on other absorbent articles. The following enhancement products 20 are among those that may be used in a diaper-based system:

An extra absorbency liner 50 is an absorbent-containing liner sized to fit within the diaper, between the diaper and a wearer of the diaper. As used herein, the term “liner” implies that the enhancement product 20 is put on the inside of the product. The liner does not need to extend over the entire width or length of the product. The extra absorbency liner 50 supplements the absorbent capacity of the chassis 15. The extra absorbency liner 50 may be inserted for overnight use of the chassis, for long car trips, or whenever extra absorbency is desired.

A BM liner 55 is designed to quickly separate BM from skin contact with the wearer to limit the exposure of the skin. The BM liner 55 is sized to fit within the diaper, between the diaper and the wearer of the diaper.

A lotion liner 60 is a liner coated, impregnated, or otherwise treated with a lotion designed to promote skin health. The lotion may be applied in stripes or other patterns to help to maintain the absorbency of the diaper. In the diaper example, the lotion may prevent or treat diaper rash. A lotion liner 60 such as this may be used consistently, or, more likely, it may be used only on occasions when diaper rash is anticipated based on stool consistency, the wearer's health, etc. Diaper rash lotions may be difficult to remove from the applier's fingers; simply inserting a lotion liner 60 would avoid such a mess. In alternate embodiments, any other suitable lotion may be used in conjunction with the lotion liner 60. Further examples include petrolatum, zinc oxide, and cornstarch.

An odor tab 65 is designed to be inserted in or attached to the chassis 15 to reduce or eliminate odors emanating from the diaper. The odor tab 65 may include baking soda and/or activated charcoal to absorb urine or BM odor. While some consumers may prefer to have some odor remain as indicator of the need to change the diaper, a reduction in odor may still be desirable for those consumers. Multiple odor tabs 65 may be used for additional odor reduction or elimination.

Similarly, an aromatherapy tab 70 is designed to be inserted in or attached to the chassis 15 to produce beneficial aromas such as lavender or chamomile to soothe and relax or peppermint to refresh the wearer, similar to the aromas produced by aromatherapy candles and bathing products.

A humidity tab 72 may be a small packet that sticks to the inside of the wasteband to help control humidity inside the diaper and keep the wearer's skin dryer.

A health screen 75 may be inserted into or attached to the chassis 15 to screen for one or more health conditions. A health screen 75 may test for one or multiple conditions.

In addition, one or more health screens 75 may be used. Examples of health screens 75 include, but are not limited to, a dehydration sensor, a rash screen, a urinary tract infection screen, and medical diagnostic screens such as indicators of sugar content in urine and urine pH. Health screens for elderly incontinent adults who are often institutionalized with a variety of medical conditions present a large opportunity for a variety of enhancement products. Health screens 75 may not be typically used on a daily basis, but may be purchased to be ready in case a health condition is suspected.

A wetness monitor 80 is designed to indicate to a caregiver when the absorbent article has been made wet. Such an indication is particularly useful in newborn's whose wet diapers are difficult to detect. The wetness monitor 80 may generate a light or sound in the presence of wetness, may transmit a signal to a monitor, may change colors or cause a graphic to appear or disappear in the presence of wetness, or may provide any other suitable indication of wetness.

An air dry liner 85 is a liner that provides additional air circulation between the chassis and the wearer's skin to promote skin health and dryness.

A bio tissue 90 is designed to be folded into a diaper prior to disposal to promote biodegradation of the diaper to minimize environmental loading due to disposable diapers.

A diaper cover 100 is worn by the wearer over and outside of the diaper and is designed to improve the fit of the diaper and to promote leakage protection. The diaper cover 100 may be a mesh cover, and the diaper cover 100 may be decorative.

Suspenders 105 may be used to help keep diapers up on babies who have a hard time keeping clothes up. The suspenders 105 may be made from a soft, stretchy material, and soft fasteners may be used to fasten the suspenders 105 to the diaper without harming the skin of the wearer.

In addition to enhancement products 20, the absorbent article system 10 may also include tertiary or associated products 140. Associated products 140 are products that would likely be used by those who use the chassis 15 and enhancement products 20. Associated products 140 may be used to enhance the use of the absorbent article system 10, to assist in the education provided by the absorbent article system 10, or for any other suitable use. In the diaper example, associated products 140 may include infant bathing products, infant toys, infant powders and ointments, changing pads, specialty wipes, and the like.

In an example of a specific application of the absorbent article system 10, a toilet training system 150 of products related to toilet training may be provided.

The chassis 15 as described herein for a toilet training system 150 may be either a diaper or a training pant. A useful enhancement product 20 in the toilet training system 150 may be a disposable shell designed to be worn over the chassis 15.

Another useful enhancement product in the toilet training system 150 is one that would provide notification to the wearer of wetness, allowing the wearing to associate the notification with certain body signals that indicate that wetness is imminent. A wetness notification liner or insert 155 may include a bicomponent spunbond web that includes a water absorbing component, a bonded carded web containing a hydrophilic material such as cotton or rayon, or a hydrophobic spunbond web that is treated with a durable wettable treatment such as a surfactant. The wetness notification liner 155 may function by cooling, warming, releasing a gas, releasing an astringent, changing texture, reducing porosity such that any liquid is absorbed more slowly into an absorbent core, releasing a scent, or changing in the z-dimension (towards the wearer) when wet, or some combination thereof. The wetness notification liner 155 may also function by including items that attach to the liner 155 or chassis 15 and produce noise or light to alert both the wearer and a caregiver of the wetness.

In another embodiment, in the toilet training system 150 example, associated products 140 may include graphics activated by oxygen or electricity that appear with time to signal how long the chassis 15 has been on the child; attachments to the wetness notification liner 155 or other liner or the chassis 15 that cause graphics to appear or disappear with wetness, stickers; progress charts; toilet training educational materials such as videos, books, and hint sheets; coloring books and/or crayons and markers; urine targets; and graphics on the shell that disappear when wet. Other associated products 140 may include bibs, changing pads, onesies, and medicated or other specialty wipes.

The absorbent article system 10 described herein may also employ as a chassis 15 a feminine hygiene product, an adult incontinence product, or other similar absorbent product. Each type of chassis 15 may be supplemented with suitable enhancement products 20 and associated products 140, including products similar to those described herein for use in conjunction with a diaper.

The absorbent article system 10 described herein allows a manufacturer to produce a chassis 15, enhancement products 20, and associated products 140 and package these separately. Each separate package of enhancement product 20 and associated product 160 may include a description of the condition intended to be addressed by that enhanced or associated product 20, 160, a description of the primary function of that enhancement or associated product 20, 160, a description of the entire absorbent article system 10, or some combination thereof. The descriptions may be placed on the packaging, in the packaging, or some combination of both. The descriptions may allow a consumer to become educated as to the conditions that typically dictate the use of an absorbent article, and the further conditions that may be associated with or experienced in conjunction with the use of an absorbent article. Such education allow a consumer to make informed decisions as to which enhancement and associated products 20, 160 to purchase such that the consumer may assemble a customized absorbent article to address to various conditions.

Consumer education may be further enhanced by providing indicia on the packaging to indicate various different but associated components of the absorbent article system. The indicia may include graphic design features, text, branding, or any other suitable indicia. For example, all of the packages may exhibit similar graphic design components and branding to identify their association, yet have different coloring and illustrations to identify their differences. These indicia may help a consumer to quickly discern which products may be needed for a particular purpose.

In other words, the absorbent article system 10 described herein allows a consumer to customize an absorbent article on an as-needed basis to address whatever conditions the wearer may be experiencing without spending more every day for an absorbent article that contains every enhancement. Consumers desire a high quality, consistent product at a good price with the ability to upgrade for certain circumstances.

The various components of the absorbent article system 10 may be sold through any retailer, including Internet retailers. Various distributors, wholesalers, hospitals, doctors' offices, and other suitable parties may also be involved. A retailer, for example, locates the various components of the absorbent article system 10, including packages of the chassis 15, packages of enhancement products 20, and packages of associated products 140, in general proximity to each other on one or more store shelves. Additionally, targeted enhancement devices allow for opportunities for advertising systems. For example, a health screen device could be co-marketed with the appropriate remedy for the health condition. This proximity allows a consumer to see the components of the absorbent article system 10 in one place, allows the consumer to read the packaging to learn about the absorbent article system 10, and allows the consumer to select those components that will address the consumer's needs.

As an example, a consumer wishes to prevent diaper rash in a baby susceptible to diaper rash because of periodic illness. The consumer also needs an ample supply of diapers for every day use. The consumer, as a part of a typical shopping trip, enters the baby aisle of a store and examines the diaper options. The consumer selects a brand of diapers, and notices other products located adjacent to and associated with these diapers. Upon closer examination, the consumer reads some of the other product packaging and learns that a simple lotion liner 60, when added to the diaper the consumer is already purchasing, may prevent diaper rash. The consumer thus purchases the package of diaper rash lotion liners 60 to have on hand for use only during those times when the baby is susceptible to diaper rash. The consumer may also purchase a package of extra absorbency liners 50 to have on hand for use only during occasional long car trips. The consumer may also purchase an infant toy and a package of specialty wipes. The consumer benefits from not needing to pay extra for an every day diaper that contains all of these features. The consumer also benefits because the various enhancement and associated products 20, 160 are located in one place, rather than being spread across various shelves or an entire store.

For a manufacturer, incorporating improvements into enhancement products rather than the basic absorbent article allows the manufacturer to make minimal changes to absorbent article machines, resulting in lower capital expenditures and optimizing manufacturing. In addition, researchers may focus on product improvements without considering the effect of the improvements on absorbent article machines. The manufacturer may contract manufacturing of secondary products and enter into joint marketing agreements with makers of related items. Enhancement products may be rapidly introduced and evaluated, and enhancement products need not be as cost sensitive as improvements incorporated into every absorbent article. Finally, customized enhancement products allow the manufacturer to more effectively meet global needs.

From a business standpoint, an absorbent article system may expand a category because enhancement products may be used with competitors' absorbent articles. Introduction of improvements in enhancement products may carry a lower risk than incorporating those improvements in an absorbent article.

Other objects and advantages of the present invention will become more apparent to those skilled in the art in view of the following description and the accompanying drawings.

The invention has been described with reference to various specific and illustrative embodiments and techniques. However, it should be understood that many variations and modifications may be made while remaining within the spirit and scope of the invention. Many alternatives, modifications and variations will be apparent to those skilled in the art in light of the foregoing description. Accordingly, this invention is intended to embrace all such alternatives, modifications, and variations that fall within the spirit and scope of the appended claims.

We claim:

1. A method for providing a system of absorbent articles, the method comprising:
offering a chassis primarily performing a base function, wherein the chassis is an absorbent article;
offering a first enhancement product primarily performing a first enhanced function; and
offering a second enhancement product primarily performing a second enhanced function, wherein the chassis and the first and second enhancement products are adapted such that the first and second enhancement products may be coupled to the chassis, and wherein the second enhanced function is different from the first enhanced function.

2. The method of claim 1, further comprising offering a third enhancement product primarily performing a third enhanced function, wherein the chassis and the third enhancement product are adapted such that the third enhancement product may be coupled to the chassis, and wherein the third enhanced function is different from the first and second enhanced functions.

3. The method of claim 1, wherein the absorbent article is a diaper.

4. The method of claim 1, wherein the absorbent article is a training pant.

5. The method of claim 1, wherein the absorbent article is a swim pant.

6. The method of claim 1, wherein the absorbent article is a youth pant.

7. The method of claim 1, wherein the absorbent article is a feminine hygiene product.

8. The method of claim 1, wherein the absorbent article is an incontinence product.

9. The method of claim 1, wherein the base function is absorption and collection of a bodily fluid.

10. The method of claim 9, wherein the bodily fluid is selected from the group consisting of urine, menses, feces, saliva, sweat, and blood.

11. The method of claim 1, wherein the first enhanced function is selected from the group consisting of absorption, odor control, air circulation enhancement, aroma provision, biodegradation, health screening, humidity control, rash protection, fit protection, leakage protection, and wetness monitoring.

12. The method of claim 1, wherein the second enhanced function is selected from the group consisting of odor control, air circulation enhancement, aroma provision, biodegradation, health screening, humidity control, rash protection, fit protection, leakage protection, and wetness monitoring.

13. The method of claim 1, wherein the first enhancement product is selected from the group consisting of a liner, a tab, an insert, a sensor, a sticker, a cover, a garment, a monitor, and a tissue.

14. The method of claim 1, wherein the second enhancement product is selected from the group consisting of a liner, a tab, an insert, a sensor, a sticker, a cover, a garment, a monitor, and a tissue.

15. The method of claim 1, wherein the absorbent article may be used alone without the first or second enhancement products.

16. A method for educating a consumer with regard to various conditions requiring an absorbent article, the method comprising:
providing a first package including a description of a first condition allowing the consumer to learn about the first condition and what may be used to address the first condition, wherein the first package includes a first enhancement product adapted to address the first condition;
providing a second package including a description of a second condition allowing the consumer to learn about the second condition and what may be used to address the second condition, wherein the second package includes a second enhancement product adapted to address the second condition, and wherein the second condition is different from the first condition; and
providing a third package including a description of a base condition allowing the consumer to learn about the base condition and what may be used to address the base condition, wherein the third package includes a chassis adapted to address the base condition and adapted such that the chassis may be combined with the first and/or second enhancement products, and wherein the base condition is different from the second condition.

17. The method of claim 16, further comprising providing a fourth package including a description of a fourth condition allowing the consumer to learn about the fourth condition and what may be used to address the fourth condition, wherein the fourth package includes a fourth enhancement product adapted to address the fourth condition, wherein the fourth condition is different from the first, second, and base conditions.

18. The method of claim 16, further comprising providing indicia on each of the first, second, and third packages indicating that the first and second enhancement products are compatible with the chassis.

19. The method of claim 16, wherein the base condition is a need for absorption and collection of a bodily fluid.

20. The method of claim 16, wherein the absorbent article is a diaper.

21. The method of claim 16, wherein the absorbent article is a training pant.

22. The method of claim 16, wherein the absorbent article is a swim pant.

23. The method of claim 16, wherein the absorbent article is a youth pant.

24. The method of claim 16, wherein the absorbent article is a feminine hygiene product.

25. The method of claim 16, wherein the absorbent article is an incontinence product.

26. A system for addressing various conditions requiring an absorbent article, the system comprising:
an absorbent chassis adapted to absorb a bodily fluid;
a first enhancement product adapted to perform a first enhanced function, wherein the chassis and the first enhancement product are adapted such that the first enhancement product may be coupled to the chassis; and
a second enhancement product adapted to perform a second enhanced function, wherein the chassis and the second enhancement product are adapted such that the second enhancement product may be coupled to the chassis, and wherein the second enhanced function is different from the first enhanced function.

27. The system of claim 26, further comprising a third enhancement product adapted to perform a third enhanced function, wherein the chassis and the third enhancement product are adapted such that the third enhancement product may be coupled to the chassis, and wherein the third enhanced function is different from the first and second enhanced functions.

28. A method for a consumer to customize an absorbent article to address a condition, the method comprising:
selecting a base absorbent article;
examining a plurality of packages, each containing an enhancement product;
selecting the enhancement product adapted to address the condition; and
combining the enhancement product and the base absorbent article.

29. A method for selling a customizable absorbent article system, the method comprising:
displaying on a store shelf a chassis, wherein the chassis is an absorbent article; and
displaying, adjacent to the chassis, a plurality of different enhancement products, wherein the chassis and each enhancement product are adapted such that each enhancement product may be coupled to the chassis.

30. The method of claim 29, further comprising displaying, adjacent to the chassis and the plurality of different enhancement products, a plurality of different associated products.

31. The method of claim 30, wherein each of the chassis, plurality of different enhancement products, and plurality of different associated products includes indicia indicating that the plurality of different enhancement products and the plurality of different associated products are compatible with the chassis.
